(19) United States
(12) Patent Application Publication
(10) Pub. No.: US 2017/0248617 A1
(43) Pub. Date: Aug. 31, 2017

(54) METHOD OF PREDICTING RESPONSE OF A HUMAN SUBJECT SUFFERING FROM MULTIPLE SCLEROSIS TO INTERFERON BETA, (IFN-ß)

(71) Applicant: FUNDACIÓN PARA LA INVESTIGACIÓN BIOMÉDICA DEL HOSPITAL UNIVERSITARIO RAMÓN Y CAJAL, Madrid (ES)

(72) Inventors: Luisa María VILLAR GUIMERANS, Madrid (ES); José Carlos ÁLVAREZ CERMEÑO, Madrid (ES)

(21) Appl. No.: 15/510,579

(22) PCT Filed: Jul. 15, 2015
(86) PCT No.: PCT/EP2015/066163

§ 371 Date: Apr. 3, 2017

(30) Foreign Application Priority Data
Sep. 11, 2014 (ES) P201431314

(51) Int. Cl. G01N 33/68; A61K 38/21

(52) U.S. Cl. CPC G01N 33/6896; A61K 38/215; A61K 47/48215; G01N 2800/285; G01N 2800/52; G01N 2333/70589; G01N 2333/70596

(57) ABSTRACT

The present invention refers to a method of predicting response of a human subject to Interferon beta, (IFN-β), wherein the subject is suffering from Multiple Sclerosis (MS), and wherein the method comprises using, as an indicator, the percentage of CD5+ CD19+ CD45+ B cells over the total count of lymphocytes (CD45+ cells) in a biological sample originating from the human subject and the percentage of CD8+ CD45+ perforin+ T cells over the total count of lymphocytes (CD45+ cells) in a biological sample originating from the human subject, wherein if the percentage of CD5+ CD19+ CD45+ B cells over the total count of lymphocytes (CD45+ cells) is lower than or equal to 3% and/orthe percentage of CD8+ CD45+ perforin+ T cells over the total count of lymphocytes (CD45+ cells) is greater than or equal to 1%, is indicative of response.

### FIELD OF THE INVENTION

The present invention relates to the medical field, particularly to a method of predicting response in a human subject suffering from Multiple Sclerosis to interferon beta.

### BACKGROUND OF THE INVENTION

Multiple sclerosis (hereinafter briefly referred to as “MS”) is a disease in which fatty sheaths known as “myelin” covering nerve fibers in the brain and spinal cord undergo inflammation, and thereby nervous information is not satisfactorily communicated, thus causing various symptoms such as visual disturbance, dyskinesia, hyposensitivity, and equilibration disorder. The cause of MS has not yet been clarified, and MS is one of chronic diseases, which the present medicine cannot cure completely. It is believed to be an autoimmune disease in which the immune system of an individual attacks oneself in error, but the detailed mechanism of its onset has not yet been clarified. It is reported that there are about one million patients with MS in the world.

One of features of MS is that most of patients with MS repeat relapse a number of times. MS is roughly classified as relapsing-remitting MS and progressive MS. In the relapsing-remitting MS, the patients undergo relatively satisfactory recovery when they have undergone an acute phase and enter a remission phase, while the magnitude and duration of the relapse vary from patient to patient. Some of the patients with relapsing-remitting MS undergo increasing after effects and progression with an increasing time of relapse. In contrast, in the progressive MS, the patients undergo gradual progression of the disease without significant recovery.

Effects to suppress the relapse of MS have been found in a genetically recombinant interferon [beta], and this substance has been considered as an effective treatment to suppress the relapse and/or progression of MS. The interferon [beta] includes interferon [beta]-1a commercially available, for example, under the trade name of ABONEX (from Biogen) and interferon [beta] 1 b commercially available, for example, under the trade name of BETAFERON (from SCHERING AG). These agents, however, invite flulike symptoms, injection-site reactions, headache, fatigue, depression, and psoriasis as adverse drug reactions. In addition, the efficacy of these agents is found in only about 20% to 30% of patients treated with the agents and is not found in the rest of the patients. Specifically, about 70% to 80% of patients treated with the interferon [beta] suffer adverse drug reactions alone without the effects of reducing the frequency of relapse and retarding of the progression of physical disorders. Strong demands have therefore been made to develop an evaluation method of the efficacy of the treatment to thereby reduce the number of patients suffering from such adverse drug reactions. Conventional evaluation methods of the efficacy include, for example, magnetic resonance imaging (MRI) tests, evoked potential tests, and spinal tap. The MRI tests can differentiate active foci from cured foci by using gadolinium as a contrast medium and are very useful, but cannot detect every focus. The evoked potential tests determine the presence or absence of a focus on the neurotransmission pathway by applying visual, somatic and/or auditory stimuli to a subject, and determining the speed and intensity of signals transmitting on the neurotransmission pathway. The spinal tap detects the presence or absence of a focus by sampling a cerebrospinal fluid flowing around the brain and spinal cord and determining the amounts of leukocytes, antibodies (immunoglobulin G; TgG) and myelin basic proteins in the spinal fluid and is very useful. However, this test requires puncture on the back of the subjects and puts an enormous load or burden on subjects. These conventional evaluation methods cannot significantly and easily evaluate the efficacy of the interferon [beta] treatment at early stages with a high detection sensitivity and less burden on the subjects.

### BRIEF DESCRIPTION OF THE INVENTION

It is highly desirable to predict which Multiple Sclerosis patients respond well to a therapeutic treatment with interferon beta. A goal of the present invention is thus the provision of an easy and reliable in vitro method for predicting progression-free survival of Multiple Sclerosis patients undergoing therapy.

Thus, a primary goal of the present invention is to overcome several drawbacks associated with Multiple Sclerosis predictive methods according to the state of the art. A particular goal addressed is the provision of methods and tools for predicting progression-free survival of Multiple Sclerosis patients. A further goal is to provide the appropriate medication for an individual Multiple Sclerosis patient, based on the prediction of the success of the treatment. A further problem to be solved is the provision of a detection system, such as a kit, which may be useful in the methods of the invention.

### BRIEF DESCRIPTION OF THE FIGURES

FIG. 1. Percentage of patients treated with interferon beta pertaining to each of the two subgroups (active disease and free of disease). It is noted that no difference were found between the different interferon beta treatments.

FIGS. 2 and 3. FIG. 2 shows the statistical analysis of the results obtained in FIG. 3. FIG. 3 shows a flow cytometry analysis showing the distribution of the different cell populations and the biomarkers associated to interferon beta response.

FIG. 4. FIG. 4 shows that a percentage of CD45+CD19+CD5+ lymphocyte cells higher than 3% predicts an active disease during interferon beta treatment.

FIG. 5. FIG. 5 shows that a percentage of CD45+CD8+perforin+CD56-CD3+ T cells lower than 3% predicts an active disease during interferon beta treatment.

### DETAILED DESCRIPTION OF THE INVENTION

The detailed description discloses specific and/or preferred variants of the individual features of the invention. The present invention also contemplates as particularly preferred embodiments those embodiments, which are generated by combining two or more of the specific and/or preferred variants described for two or more of the features of the present invention.

The authors of the present invention have surprisingly discovered that low percentages of CD45+CD19+CD5+ cells and high percentages of CD45+CD8+perforin+CD56-CD3+ T cells permit the identification of good responders to interferon beta therapy in Multiple Sclerosis patients before treatment initiation.

In this sense, the authors of the present invention in order to reach the above conclusion recruited 119 consecutive patients suffering from relapsing remitting multiple sclerosis (RRMS). All of these patients initiated interferon beta treatment (Avonex or Rebif or betaferon) at Ramón y Cajal hospital (Madrid, Spain). From the 119 original patients recruited, only 99 of these patients completed a 2-years follow-up procedure. During this follow-up procedure the patients were monitored for the appearance of new relapses, disability progression and new lesions on annual MRI scans.

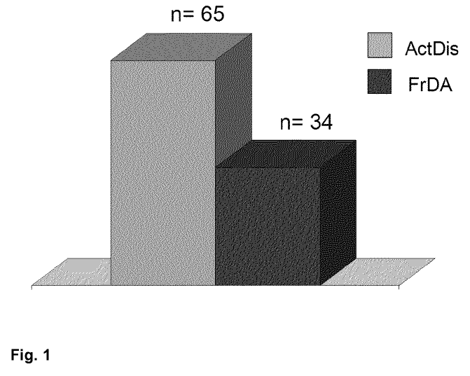

As illustrated in FIG. 1, of the 99 patients recruited, 65 of them showed active disease (ActDis) (namely 65.7% of the total number of patients which completed the 2 years follow-up procedure showed clinical signs of the disease after the two years) and 34.3% of the patients were free of disease activity (FrDA) (no new relapses nor disability progression or new lesions on MRI scans after the two years follow-up procedure). It is also important to note that 9 patients of the 65 patients having active disease after the two-year follow-up procedure developed neutralizing antibodies anti-interferon beta.

Before initiating treatment with interferon beta, different factors (soluble factors (table 1) and leukocyte subsets (tables 2 to 4)) were analysed by the authors of the present invention for each of the patients recruited. The following tables establish each of the studied factors.

TABLE 1
Soluble molecules: concentration of cytokines and vitamins
before treatment initiation in both subgroups (active
disease and free of disease)

| | | |
|---|---|---|
| FrDA | ActDis | P |

Cytokines

| | | | |
|---|---|---|---|
| IL-12 (pg/ml) | 12.91 ± 7.53 | 9.23 ± 7.9 | ns |
| IFN-γ (pg/ml) | 12.81 ± 3.98 | 8.31 ± 3.8 | ns |
| IL-17F (pg/ml) | 30.56 ± 10.85 | 44.12 ± 19.12 | ns |
| IL-1β (pg/ml) | 17.49 ± 6.59 | 5.29 ± 3.58 | ns |
| TNF-α (pg/ml) | 23.21 ± 6.73 | 20.76 ± 9.71 | ns |
| IL-2 (pg/ml) | 11.48 ± 4.96 | 12.86 ± 7.14 | ns |
| IL-10 (pg/ml) | 14.29 ± 4.22 | 13.54 ± 6.78 | ns |
| IL-22 (pg/ml) | 396.5 ± 82.36 | 350.9 ± 92.33 | ns |
| IL-6 (pg/ml) | 7.65 ± 2.13 | 8.82 ± 2.93 | ns |
| IL-13 (pg/ml) | 14.08 ± 5.58 | 25.65 ± 10.23 | ns |
| IL-4 (pg/ml) | 39.29 ± 10.03 | 75.07 ± 32.76 | ns |
| IL-5 (pg/ml) | 22.04 ± 8.3 | 45.27 ± 18.56 | ns |
| Activin-A | 141.5 ± 55.19 | 126.8 ± 43.91 | ns |

Vitamins

| | | | |
|---|---|---|---|
| Vitamin D | 14.84 ± 1.02 | 17.64 ± 1.82 | ns |
| (ng/ml) | | | |
| VitaminA | 42.38 ± 1.96 | 45.98 ± 2.33 | ns |
| (μg/dl) | | | |
| Vitamin E | 973.6 ± 52.39 | 901.4 ± 6.02 | ns |
| (μg/ml) | | | |

TABLE 2
Comparative data between the different B cell surface markers before
treatment initiation among the two different subgroups (active
disease and free of disease). The results allow the selection of
those biomarkers having a significant statistical difference between
the two said subgroups. Biomarkers for which a statistical significance
has been found are highlighted in bold.

| B cells | p |
|---|---|
| CD19+ B cells | 0.001 |
| CD19+CD5+ B cells | 0.0002 |
| CD19+CD5− B cells | 0.01 |
| CD19+CD38+ B cells | 0.06 |
| CD19+CD80+ B cells | 0.24 |
| CD 19+CD86+ B cells | 0.59 |
| CD19+TNF-α B cells | 0.32 |
| CD19+IL-10 B cells | 0.53 |

TABLE 3
Comparative data between the different CD4+ T cell surface
markers before treatment initiation among the two different subgroups
(active disease and free of disease). The results allow the selection
of those biomarkers having a significant statistical difference
between the two said subgroups. Biomarkers for which a statistical
significance has been found are highlighted in bold.

| CD4+ T cells | p |
|---|---|
| CD4+ T cells | 0.63 |
| CD25hi+FoxP3+ T cells | 0.013 |
| CD4+CD25i+ T cells | 0.24 |
| CD4+TNF-α T cells | 0.38 |
| CD4+INF-γ T cells | 0.82 |
| CD4+IL-17 T cells | 0.67 |
| CD4+IL-10 T cells | 0.31 |

TABLE 4
Comparative data between the different CD8+ T cell surface
markers before treatment initiation among the two different subgroups
(active disease and free of disease). The results allow the selection
of those biomarkers having a significant statistical difference
between the two said subgroups. Biomarkers for which a statistical
significance has been found are highlighted in bold.

| CD8+ T cells | p |
|---|---|
| CD8+ T cells | 0.3 |
| CD8+Perf+ T cells | 0.006 |
| CD8+TNF-α T cells | 0.62 |
| CDS+INF-γ T cells | 0.91 |
| CD8+IL-17 T cells | 0.56 |
| CD8+IL-10 T cells | 0.65 |

From tables 2 and 4 above, the authors of the invention inferred that two specific biomarkers were associated with interferon beta good response, namely the percentage of CD5+CD19+ B cells over the total count of lymphocytes (CD45+ cells) in a biological sample of whole peripheral blood originating from a human subject suffering from Multiple Sclerosis and the percentage of CD45+CD8+perforin+CD56-CD3+ T cells over the total count of lymphocytes (CD45+ cells) in a biological sample of whole peripheral blood originating from a human subject suffering from Multiple Sclerosis.

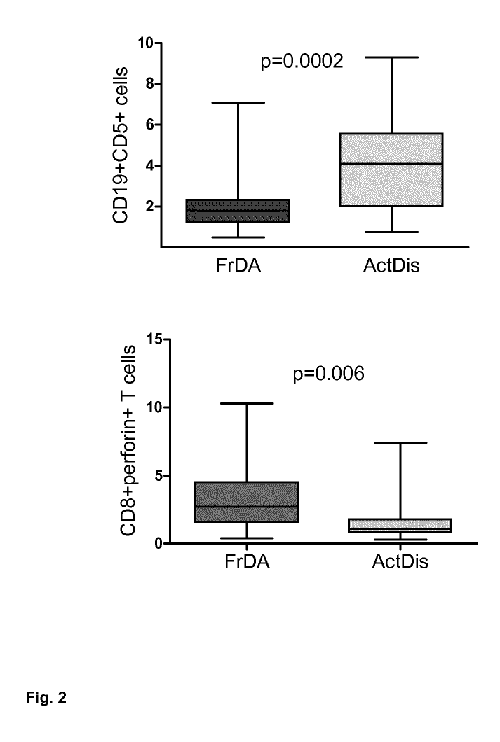

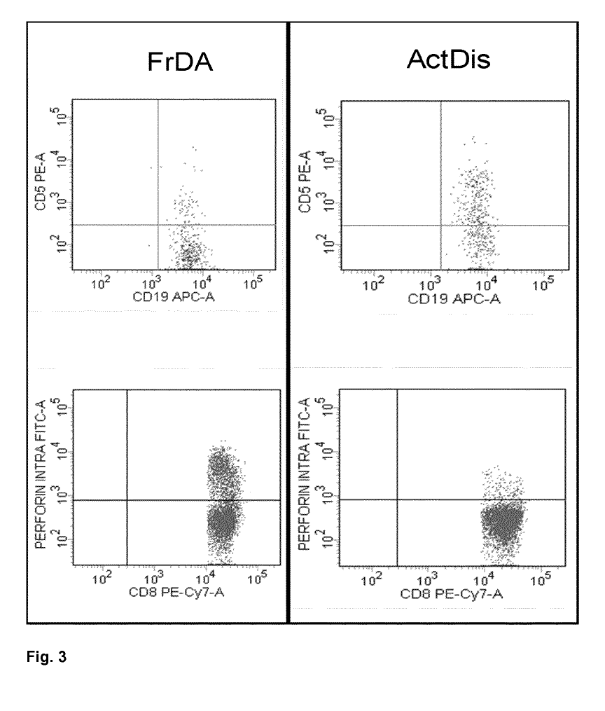

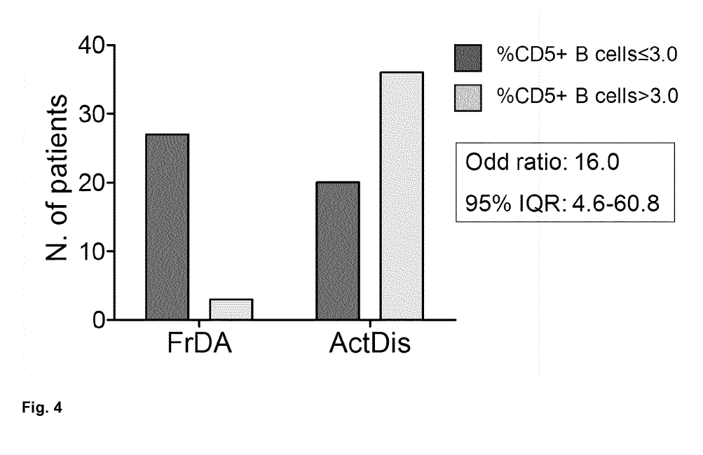

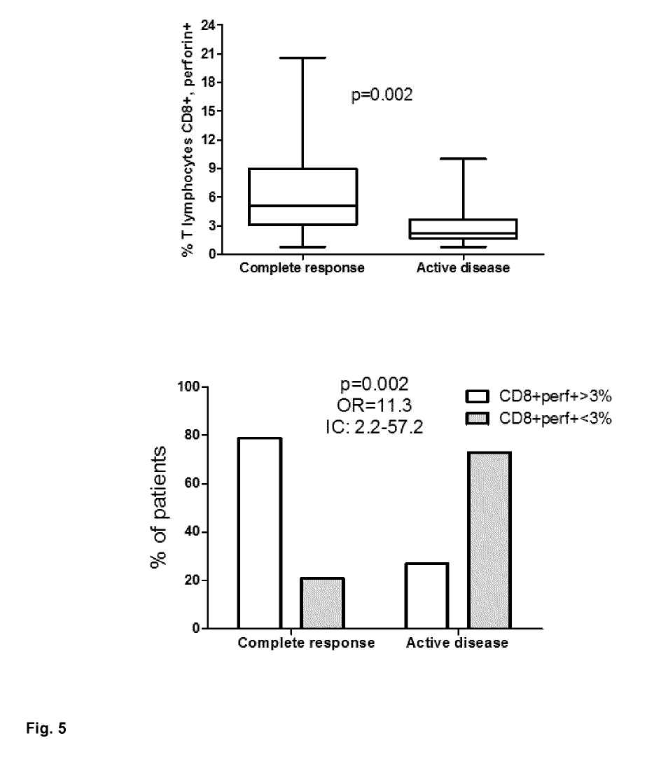

In this sense, FIGS. 2 to 4 illustrate that i) percentages of CD5+CD19+ B cells over the total count of lymphocytes (CD45+ cells) lower than or equal to 3% and/or ii) percentages of CD45+CD8+perforin+CD56-CD3+ T cells over the total count of lymphocytes (CD45+cells) greater than or equal to 1% or preferably greater than or equal to 3% (see FIG. 5) in a biological sample of whole peripheral blood originating from a human subject suffering from Multiple Sclerosis, before treatment initiation with interferon beta, are indicative of good response.

Provided in the present invention is thus a method of predicting response of a human subject to Interferon beta, (IFN-β), wherein the subject is suffering from Multiple Sclerosis (MS), and wherein the method comprises using, as an indicator, the percentage of CD5+CD19+CD45+ B cells over the total count of lymphocytes (CD45+ cells) in a biological sample originating from the human subject and the percentage of CD45+CD8+perforin+CD56-CD3+ T cells over the total count of lymphocytes (CD45+ cells) in a biological sample originating from the human subject, wherein if the percentage of CD5+CD19+CD45+ B cells over the total count of lymphocytes (CD45+ cells) is lower than or equal to 3% and/or the percentage of CD45+CD8+perforin+CD56-CD3+ T cells over the total count of lymphocytes (CD45+ cells) is greater than or equal to 1%, is indicative of response.

In a preferred embodiment, said biological sample is whole peripheral blood.

In another preferred embodiment, the percentage of CD45+CD8+perforin+CD56-CD3+ T cells over the total count of lymphocytes (CD45+ cells) is greater than or equal to 3% (see FIG. 5).

In another preferred embodiment, the response refers to the clinical outcome of the subject, more preferably to the progression-free-survival.

In another preferred embodiment, the percentages of CD45+CD8+perforin+CD56-CD3+ T cells and/or CD5+CD19+CD45+ B cells are obtainable by using flow cytometry. However, different methods to determine said percentages of cells, based or not in the flow cytometry technique, are well known in the art as illustrated in any of the following references (Ritu Gupta, M D et al, “Flow cytometric analysis of CD5+ B cells”, Hematopathology/CD5+ B-Cell Analysis by Flow Cytometry; de Jager C P C, Gemen E F A, Leuvenink J, Hilbink M, Laheij R J F, et al. (2013) Dynamics of Peripheral Blood Lymphocyte Subpopulations in the Acute and Subacute Phase of Legionnaires' Disease. PLoS ONE 8(4): e62265. doi:10.1371/journal.pone.0062265; Jorge Correale et al, “Isolation and characterization of CD8+ regulatory T cells in multiple sclerosis”, Journal of Neuroimmunology 195 (2008) 121-134; Giovanni Frisullo et al, “Circulating CD8+CD56-perforin+ T cells are increased in multiple sclerosis patients”, Journal of Neuroimmunology 240-241 (2011) 137-141; Yoshinobu Okuda et al, “The activation of memory CD4+ T cells and CD8+ T cells in patients with multiple sclerosis”, Journal of the Neurological Sciences 235 (2005) 11-17; Philip L. De Jager et al, “Cytometric profiling in multiple sclerosis uncovers patient population structure and a reduction of CD8low cells”, Brain. 2008 July; 131(Pt 7): 1701-1711. doi:10.1093/brain/awn118). Thus, the present invention is not limited to any specific manner of determining said percentages of cells.

In another preferred embodiment of the invention, the method is performed in vitro using a biological sample originating from the human subject, and wherein at the time point of taking the sample from the human subject, the human subject has not initiated treatment with interferon beta (IFN-β). Preferably, the subject has not initiated any other pharmacological treatment for treating Multiple Sclerosis or the subject has not been medicated for Multiple Sclerosis for at least one month, preferably for at least two months.

In another preferred embodiment of the invention, the interferon beta is selected from the list consisting of interferon beta-1a, interferon beta-1b, pegylated interferon beta-1a, pegylated interferon beta-1b as well as any combination thereof and other presentations of interferon beta-1.

In the present invention, the method may be performed in subjects with any stage of Multiple Sclerosis. It is noted that the profile determined by the present invention is predictive rather than prognostic and diagnostic. The subjects the response of which is predicted are human subjects suffering from Multiple Sclerosis. The terms “human subject”, “subject” and “patient” are therefore used interchangeably in this specification.

“One or more” also as used herein includes one and the individualized specification of any number which is more than one, such as two, three, four, five, six etc. “More than one” or “several” as used herein includes the individualized specification of any number which is more than one, such as two, three, four, five, six etc.

The method of the invention comprises the detection, in a sample from a human subject, of the percentage of CD5+CD19+CD45+ B cells over the total count of lymphocytes (CD45+ cells) in said biological sample and the percentage of CD45+CD8+perforin+CD56-CD3+ T cells over the total count of lymphocytes (CD45+ cells) in said biological sample. Illustrative non-limiting examples of said sample include different types of samples from tissues, as well as from biological fluids, such as blood, whole peripheral blood, plasma, cerebrospinal fluid, peritoneal fluid. Preferably, said samples are samples comprising whole peripheral blood.

Unless expressly specified otherwise, the term “comprising” is used in the context of this document to indicate that further members may optionally be present in addition to the members of the list introduced by “comprising”. It is, however, contemplated as a specific embodiment of the present invention that the term “comprising” encompasses the possibility of no further members being present, i.e. for the purpose of this embodiment “comprising” is to be understood as having the meaning of “consisting of”.

In the context of the present invention, response refers to the clinical outcome of the subject. Thus, by the method of the invention, an individual patient can be predicted to show either (i) response (R) to treatment with interferon beta or (ii) non-response (NR) to treatment with interferon beta. In the context of the present invention, “Response” is expressed as the clinical outcome, which is “progression-free of disease activity”. Progression-free of disease activity (PFA) is the length of time during and after medication or treatment during which the Multiple Sclerosis being treated by interferon beta does not progress with disease activity (such as new relapses, new lesions on MRI scans or disability progression). The decisive question is whether the individual suffering from multiple sclerosis shows clinical signs of progression of the disease after initiating treatment and preferably during 2 or more years after initiating treatment. In this sense, the present inventors have shown that after two years of follow-up, those patients having a percentage of CD5+CD19+CD45+ B cells over the total count of lymphocytes (CD45+ cells) lower than or equal to 3% and/or a percentage of CD45+CD8+perforin+CD56-CD3+ T cells over the total count of lymphocytes (CD45+ cells) greater than or equal to 3%, are free of any clinical sign of the disease.

In the context of this specification, the term “treatment” or “treating” means the administration of interferon beta, preferably interferon beta 1, to prevent, relieve or eliminate Multiple Sclerosis or of one or more symptoms associated with said disease. “Treatment” also includes preventing, relieving or eliminating the physiological sequelae of the disease. In the context of this invention, the term “relieve” is understood to mean any improvement of the situation of the treated patient—both subjectively (feelings of or about the patient) and objectively (measured parameters such as new lesions on MRI scans, new relapses and disability progression).

The method of the present invention may be applied with samples from individuals of either sex, i.e. men or women, and at any age. In particular embodiments, the individuals subjected to the method of the present invention are individuals from groups known for higher predisposition of Multiple Sclerosis.

In a second aspect, the invention also provides a method for allocating a human subject suffering from Multiple Sclerosis in one of two groups. Unless implicitly or explicitly specified otherwise, the details of the invention as detailed above also apply to the second aspect of the invention. Group 1 comprises subjects identifiable by the method of the invention as detailed above to show response; and wherein group 2 represents the remaining subjects. It is possible to provide a customized therapy to an individual, depending on whether the individual is allocated to group 1 or group 2. This is typically done as follows: (a) patients suffering from Multiple Sclerosis undergo the extraction of whole peripheral blood, (b) from said sample analysis for allocation into group 1 or group 2 is conducted. Based on allocation into group 1 or group 2, a customized therapy which may be particularly beneficial for the patient can be selected.

The present inventors have thus identified a novel subgroup of patients suffering from Multiple Sclerosis that will profit from treatment with interferon beta. Thus, the present invention also provides a pharmaceutical composition comprising an interferon beta preferably selected from the list consisting of interferon beta-1a, interferon beta-1b, pegylated interferon beta-1a and pegylated interferon beta-1b and optionally a further agent, for treating a human subject of group 1 as identifiable by the method of the invention. Given the positive prediction, the invention thus provides a method for selecting patients (group 1) which can particularly profit from administration of such a therapy. Further, it may not be justified to subject patients of group 1 to the potentially toxicity of an alternative treatment.

Further, the invention provides a pharmaceutical composition for treating a human subject of group 2 as identifiable by the method of claim 8, wherein the pharmaceutical composition comprises an active agent selected from the list consisting of: glatiramer acetate, dimethyl fumarate, fingolimod, teriflunomide, mitoxantrone and natalizumab as well as other non-interferon beta treatments suitable for the treatment of Multiple Sclerosis.

The present invention also provides a kit for predicting response of a human subject to Interferon beta, (IFN-β), comprising means for determining the percentage of CD5+CD19+CD45+ B cells over the total count of lymphocytes (CD45+ cells) in a biological sample of whole peripheral blood, and/or the percentage of CD45+CD8+perforin+CD56-CD3+ T cells over the total count of lymphocytes (CD45+ cells) in a biological sample of whole peripheral blood.

In a preferred embodiment of the invention, the means for determining the percentage of CD5+CD19+CD45+ B cells comprises the following labelled monoclonal antibodies: anti-CD5, anti-CD19 and anti-CD45 and the means for determining the percentage of CD45+CD8+perforin+CD56-CD3+ T cells comprises the following labelled monoclonal antibodies: anti-CD45, anti-CD56, anti-CD3, anti-CD8 and anti-human perforin monoclonal antibodies. Preferably, the kit further comprises all reagents (buffers, solutions, dyes . . . ) needed to implement the method of the invention such as for example for sample washing, lysing and staining. More preferably, these reagents are as defined in any of the embodiments below.

In a preferred embodiment of the invention, the means for determining the percentage of CD5+CD19+CD45+ B cells comprises at least one or all of the following labelled monoclonal antibodies: anti-CD5-PE, anti-CD19-PerCP-Cy5.5, and anti-CD45-FITC. More particularly, the means for determining the percentage of CD5+CD19+CD45+ B cells comprises at least one or all of the following labelled monoclonal antibodies: anti-CD19-PE-Cy7, anti-CD5-APC and anti-CD45-APC-H7.

It is particularly noted that different markers can be used to label the antibodies. However, preferred markers are PE also known as phycoerythrin; Cy7, APC also known as allophycocyanin, H7 and PercP.

In a more preferred embodiment of the invention, the means for determining the percentage of CD5+CD19+CD45+ B cells comprises the following tubes or recipients:
a) A tube or recipient comprising the following monoclonal antibodies anti-CD19, anti-CD5 and anti-CD45, or several tubes or recipients comprising separately or in combination each of these antibodies, wherein preferably each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μ;
b) Optionally a tube or recipient comprising monoclonal antibody anti-CD19, anti-CD3 and anti-CD45, or several tubes or recipients comprising separately or in combination each of these antibodies, wherein preferably each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μl; and
c) Optionally a lysing Solution and/or a washing or buffer solution.

It is noted that preferably, monoclonal antibody anti-CD19 is obtained from clone SJ25C1; preferably monoclonal antibody anti-CD5 is obtained from clone L17F12, preferably monoclonal antibody anti-CD3 is obtained from clone SK7 and preferably monoclonal antibody anti-CD45 is obtained from clone 2D1. Each of these clones are well known in the art and each of these monoclonal antibodies are publicly available.

In a more preferred embodiment of the invention, the means for determining the percentage of CD5+CD19+CD45+ B cells comprises the following tubes or recipients:
a) A tube or recipient comprising labelled monoclonal antibody anti-CD19-PE-Cy7, anti-CDS-APC and anti-CD45-APC-H7, or several tubes or recipients comprising separately or in combination each of these antibodies; and
b) Optionally a lysing Solution and/or a washing or buffer solution.

More preferably, the means for determining the percentage of CD5+CD19+CD45+ B cells comprises the following tubes or recipients:
a. A tube or recipient comprising labelled monoclonal antibody anti-CD19-PE-Cy7, anti-CD5-APC and anti-CD45-APC-H7, or several tubes or recipients comprising separately or in combination each of these antibodies, wherein each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μl; and
b. Optionally a lysing Solution and/or a washing or buffer solution.

In a more preferred embodiment of the invention, the means for determining the percentage of CD5+CD19+CD45+ B cells comprises the following tubes or recipients:
a) A tube or recipient comprising labelled monoclonal antibody anti-CD19-PE-Cy7, anti-CD3-APC and anti-CD45-APC-H7, or several tubes or recipients comprising separately or in combination each of these antibodies;
b) A tube or recipient comprising labelled monoclonal antibody anti-CD19-PE-Cy7, anti-CD5-APC and anti-CD45-APC-H7, or several tubes or recipients comprising separately or in combination each of these antibodies; and
c) Optionally a lysing Solution and/or a washing or buffer solution.

More preferably, the means for determining the percentage of CD5+CD19+CD45 B cells comprises the following tubes or recipients:
a) A tube or recipient comprising labelled monoclonal antibody anti-CD19-PE-Cy7, anti-CD3-APC and anti-CD45-APC-H7, or several tubes or recipients comprising separately or in combination each of these antibodies, wherein each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μl;
b) A tube or recipient comprising labelled monoclonal antibody anti-CD19-PE-Cy7, anti-CD5-APC and anti-CD45-APC-H7, or several tubes or recipients comprising separately or in combination each of these antibodies, wherein each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μl; and
c) Optionally a lysing Solution and/or a washing or buffer solution.

In a preferred embodiment of the invention, the means for determining the percentage of CD8+CD45+perforin+ T cells comprises at least one or all of the following monoclonal antibodies: anti-CD8, anti-CD3, anti-CD56, anti-CD45 and Anti-Perforin Antibody, preferably FITC Anti-human Perforin Antibody.

In a more preferred embodiment of the invention, the means for determining the percentage of CD45+CD8+perforin+CD56-CD3+ T cells comprises the following tubes or recipients:
a. A tube or recipient comprising anti-CD8, anti-CD3, anti-CD56 and anti-CD45, or several tubes or recipients comprising separately or in combination each of these antibodies;
b. A tube or recipient comprising an Anti-Perforin Antibody, preferably FITC Anti-human Perforin Antibody; and
c. Optionally means for the fixation and permeabilization of cells, which is necessary for staining intracellular cytokines with fluorochrome-conjugated anti-cytokine antibodies. For this purpose the kit may comprise two reagents, a fixation/permeabilization solution and a buffer solution. After cell fixation and permeabilization, the buffer is used to wash the cells and to dilute the anti-cytokine antibodies for staining.

In a more preferred embodiment of the invention, the means for determining the percentage of CD45+CD8+perforin+CD56-CD3+ T cells comprises the following tubes or recipients:
a. A tube or recipient comprising labelled monoclonal antibodies anti-CD8-PE, anti-CD3-PercP, anti-CD56-APC and anti-CD45-APC-H7, or several tubes or recipients comprising separately or in combination each of these antibodies;
b. A tube or recipient comprising an Anti-Perforin Antibody, preferably FITC Anti-human Perforin Antibody; and
c. Optionally means for the fixation and permeabilization of cells, which is necessary for staining intracellular cytokines with fluorochrome-conjugated anti-cytokine antibodies. For this purpose the kit may comprise two reagents, a fixation/permeabilization solution and a buffer solution. After cell fixation and permeabilization, the buffer is used to wash the cells and to dilute the anti-cytokine antibodies for staining.

In a more preferred embodiment of the invention, the means for determining the percentage of CD45+CD8+perforin+CD56-CD3+ T cells comprises the following tubes or recipients:
a. A tube or recipient comprising labelled monoclonal antibodies anti-CD8-PE, anti-CD3-PercP, anti-CD56-APC, anti-CD45-APC-H7 and optionally an isotype control as for example IgG_{2b}-FITC (Anti dansyl), or several tubes or recipients comprising separately or in combination each of these antibodies;
b. A tube or recipient comprising an Anti-Perforin Antibody, preferably FITC Anti-human Perforin Antibody;
c. A tube or recipient comprising anti-CD8-PE, anti-CD3-PercP, anti-CD56-APC and anti- CD45-APC-H7, or several tubes or recipients comprising separately or in combination each of these antibodies; and
d. Optionally means for the fixation and permeabilization of cells, which is necessary for staining intracellular cytokines with fluorochrome-conjugated anti-cytokine antibodies. For this purpose the kit may comprise two reagents, a fixation/permeabilization solution and a buffer solution. After cell fixation and permeabilization, the buffer is used to wash the cells and to dilute the anti-cytokine antibodies for staining.

More preferably, the means for determining the percentage of CD45+CD8+perforin+CD56-CD3+ T cells comprises the following tubes or recipients:
a. A tube or recipient comprising monoclonal antibodies anti-CD8-PE, anti-CD3-PercP, anti-CD56-APC and anti-CD45-APC-H7, or several tubes or recipients comprising separately or in combination each of these antibodies, wherein each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μl;
b. A tube or recipient comprising Anti-Perforin Antibody, preferably FITC Anti-human Perforin Antibody; and
c. Optionally an isotype control as for example IgG_{2b}-FITC (Anti dansyl) and means for the fixation and permeabilization of cells, which is necessary for staining intracellular cytokines with fluorochrome-conjugated anti-cytokine antibodies. For this purpose the kit may comprise two reagents, fixation/permeabilization solution and a buffer solution. After cell fixation and permeabilization, the buffer is used to wash the cells and to dilute the anti-cytokine antibodies for staining.

More preferably, the means for determining the percentage of CD45+CD8+perforin+CD56-CD3+ T cells comprises the following tubes or recipients:
a. A tube or recipient comprising monoclonal antibodies anti-CD8-PE, anti-CD3-PercP, anti-CD56-APC, anti-CD45-APC-H7 and optionally an isotype control as for example IgG_{2b}-FITC (Anti dansyl), or several tubes or recipients comprising separately or in combination each of these antibodies, wherein each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μl;
b. A tube or recipient comprising an Anti-Perforin Antibody, preferably FITC Anti-human Perforin Antibody; and
c. A tube or recipient comprising labelled monoclonal antibody anti-CD8-PE, anti-CD3-PercP, anti-CD56-APC and anti-CD45-APC-H7, or several tubes or recipients comprising separately or in combination each of these antibodies, wherein each of these monoclonal antibodies is found at a concentration between 0.02 and 1.5 μg/μl, preferably between 0.05 and 1 μg/μl, preferably between 0.1 and 0.5 μg/μl, more preferably about 0.2 μg/μl; and
d. Optionally an isotype control as for example IgG_{2b}-FITC (Anti dansyl) and means for the fixation and permeabilization of cells, which is necessary for staining intracellular cytokines with fluorochrome-conjugated anti-cytokine antibodies. For this purpose the kit may comprise two reagents, fixation/permeabilization solution and a buffer solution. After cell fixation and permeabilization, the buffer is used to wash the cells and to dilute the anti-cytokine antibodies for staining.

The kits disclose herein are based on the predictive power of the method of the present invention. As said above, reference value indicative for response for each particular percentage has been clearly established above. In the particular case of the kit, the reference value indicative for non-response (and/or a reference value indicative for response) may be provided with the kit. With the help of the kit, the percentages of each of the cell types can be calculated.

A further aspect of the invention refers to the use of the kit described above for predicting response of a human subject to Interferon beta, particularly for predicting response of a human subject to Interferon beta-1, more particularly for predicting response of a human subject to Interferon beta-1a or beta-1b.

The following examples merely illustrate the present invention.

### EXAMPLES

### Example 1

### Preparing the Samples for the Determination of the Percentage of CD5+CD19+CD45+ B Cells by Flow-Cytometry

Introducing 50 μl of heparinized whole whole peripheral blood into two polystyrene tubes of 12×75 mm (sample tubes). Adding to each sample tube a mixture of monoclonal antibodies to be used in the determination of surface antigens. Said mixture comprising labelled monoclonal antibodies: anti-CD19-PE-Cy7, anti-CD3-APC and anti-CD45-APC-H7, wherein each of these monoclonal antibodies is found at a concentration of about 0.2 μg/μl. Incubating the sample tubes in the dark at room temperature for 20 minutes.

Adding 1.5 ml of FACS Lysing Solution (diluted 1/10 in distilled water) to each sample tube to remove erythrocytes. Incubating for 10 minutes in the dark at room temperature and centrifuging the sample tubes at 300 g for 7 minutes, to remove lysed red blood cells. Decanting the supernatant and re-suspending it in the residual volume.

Adding 3 ml of saline serum per sample tube and centrifuging at 300 g for 7 minutes. Decanting the supernatant and re-suspending in the residual volume.

Adding another 3 ml of saline serum per sample tube and centrifuging at 300 g for 7 minutes. Decanting the supernatant and re-suspending it in 300-400 pi of saline solution. Keeping the sample tubes at 4 degrees and in the dark until the analysis by using the flow cytometer is carrry-out (the analysis should be carried out within a maximum period of 1 h).

### Example 2

### Preparing the Samples for the Determination of the Percentage of CD45+CD8+perforin+CD56-CD3+ T Cells by Flow-Cytometry

Introducing 50 μl of heparinized whole peripheral blood into two polystyrene tubes of 12×75 mm (sample tubes). Adding to each sample tube a mixture of monoclonal antibodies to be used in the determination of surface antigens. Said mixture comprising labelled monoclonal antibodies: anti-CD8-PE, anti-CD3-PercP, anti-CD56-APC and anti-CD45-APC-H7, wherein each of these monoclonal antibodies is found at a concentration of about 0.2 μg/μl;

Incubating the sample tubes in the dark at room temperature for 20 minutes. Adding 3 ml of saline serum per sample tube and centrifuging at 300 g for 7 minutes. Decanting the supernatant and re-suspendeding it in the residual volume.

Adding 200 ul of Cytofix/Cytoperm (or any other fixation/permeabilization solution), stir in the vortex and incubate for 20 min at 4° C. and in darkness. Adding 2 ml of Perm/Wash Buffer (1/10 dilution in distilled water) (or of any other buffer solution) per tube and centrifuging at 300 g for 7 minutes. Decanting the supernatant and re-suspending it in the residual volume.

Adding to the corresponding sample tube the proper amount of IgG2b anti-dansyl and to the other sample tube the proper amount of FITC Anti-human Perforin Antibody.

Vortex and incubate for 30 minutes at 4° C. and in darkness.

Adding 2 ml of Perm/Wash Buffer (1/10 dilution in distilled water) (or of any other buffer solution) per sample tube and centrifuging at 300 g for 7 minutes. Decanting the supernatant and re-suspending it in 300-400 μl of Perm/Wash Buffer (1/10 dilution in distilled water).

Keeping the sample tubes at 4° C. and in the dark until analyzed in the flow cytometer (the analysis should be carried out within a maximum period of 1 h).

1. An in vitro method of predicting response of a human subject to Interferon beta, (IFN-β), wherein the subject is suffering from Multiple Sclerosis (MS), and wherein the method comprises using, as an indicator, the percentage of CD5+ CD19+ CD45+ B cells over the total count of lymphocytes (CD45+ cells) in a biological sample originating from the human subject, wherein if the percentage of CD5+ CD19+ CD45+ B cells over the total count of lymphocytes (CD45+ cells) is lower than or equal to 3%, is indicative of response.

2. The in vitro method of claim 1, which further comprises using, as an indicator, the percentage of CD45+CD8+perforin+CD56-CD3+ T cells over the total count of lymphocytes (CD45+ cells) in a biological sample originating from the human subject, wherein if the percentage of CD5+ CD19+ CD45+ B cells over the total count of lymphocytes (CD45+ cells) is lower than or equal to 3% and the percentage of CD8+ CD45+ perforin+ T cells over the total count of lymphocytes (CD45+ cells) is greater than or equal to 1%, is indicative of response.

3. The method of any of claim 1 or 2, wherein the biological sample is whole peripheral blood.

4. The method according to any of claim 2 or 3, wherein the percentage of CD8+ CD45+ perforin+ T cells over the total count of lymphocytes (CD45+ cells) is greater than or equal to 3%.

5. The method according to any of the precedent claims, wherein response refers to the progression-free of disease activity after initiating treatment.

6. The method according to any of the precedent claims, wherein the percentage of CD5+ CD19+ CD45+ B cells over the total count of lymphocytes (CD45+ cells) in a biological sample originating from the human subject and the percentage of CD8+ CD45+ perforin+ T cells over the total count of lymphocytes (CD45+ cells) in a biological sample originating from the human subject is obtained by flow cytometry.

7. The method according to any one of the preceding claims, wherein, the method is performed in vitro using a biological sample originating from the human subject, and wherein at the time point of taking the sample from the human subject, the human subject has not initiated treatment with interferon beta (IFN-β).

8. The method of any one of the preceding claims, wherein the interferon beta is interferon beta-1a and/or interferon beta-1b and/or pegylated interferon beta-1a and/or pegylated interferon beta-1b.

9. A method for allocating a human subject suffering from Multiple Sclerosis in one of two groups, wherein group 1 comprises subjects identifiable as responders by the method according to claims 1 to 8, and wherein group 2 represents the remaining subjects.

10. A pharmaceutical composition comprising an interferon beta preferably selected from the list consisting of interferon beta-1a, interferon beta-1b, pegylated interferon beta-1a and pegylated interferon beta-1b and optionally a further agent, for treating a human subject of group 1 as identifiable by the method of claim 9.

11. A pharmaceutical composition for treating a human subject of group 2 as identifiable by the method of claim 9, wherein the pharmaceutical composition comprises an active agent selected from the list consisting of: glatiramer acetate, dimethyl fumarate, fingolimod, teriflunomide, mitoxantrone and natalizumab.

12. A kit comprising means for determining the percentage of CD5+CD19+CD45+ B cells over the total count of lymphocytes (CD45+ cells).

13. The kit of claim 12, which further comprises means for determining the percentage of CD45+CD8+perforin+CD56-CD3+ T cells over the total count of lymphocytes (CD45+ cells).

14. The kit of claim 12 or 13, wherein the means for determining the percentage of CD5+CD19+CD45+ B cells comprises the following labelled monoclonal antibodies: anti-CD19-PE-Cy7, anti-CD5-APC, anti-CD45-APC-H7 and optionally anti-CD3-APC.

15. The kit of claim 12 or 13, wherein the means for determining the percentage of CD5+CD19+CD45+ B cells comprises the following tubes or recipients:
a. A tube or recipient comprising the following monoclonal antibodies anti-CD19, anti-CD5 and anti-CD45, or several tubes or recipients comprising separately or in combination each of these antibodies, wherein preferably each of these monoclonal antibodies is found at a concentration between 0.02 and 1.5 μg/μl;
b. Optionally a tube or recipient comprising monoclonal antibody anti-CD19, anti-CD3 and anti-CD45, or several tubes or recipients comprising separately or in combination each of these antibodies, wherein preferably each of these monoclonal antibodies is found at a concentration between 0.02 and 1.5 μg/μl; and
c. Optionally a lysing Solution and/or a washing or buffer solution.

16. The kit of claim 12 or 13, wherein the means for determining the percentage of CD5+CD19+CD45+ B cells comprises the following tubes or recipients:
a. A tube or recipient comprising labelled monoclonal antibody anti-CD19-PE-Cy7, anti-CD5-APC and anti-CD45-APC-H7, or several tubes or recipients comprising separately or in combination each of these antibodies; and
b. Optionally a lysing Solution and/or a washing or buffer solution.

17. The kit of claim 12 or 13, wherein the means for determining the percentage of CD5+CD19+CD45+ B cells comprises the following tubes or recipients:
a. A tube or recipient comprising labelled monoclonal antibody anti-CD19-PE-Cy7, anti-CD5-APC and anti-CD45-APC-H7, or several tubes or recipients comprising separately or in combination each of these antibodies, wherein each of these monoclonal antibodies is found at a concentration between 0.02 and 1.5 μg/μl; and
b. Optionally a lysing Solution and/or a washing or buffer solution.

18. The kit of claim 12 or 13, wherein the means for determining the percentage of CD5+CD19+CD45+ B cells comprises the following tubes or recipients:
a. A tube or recipient comprising labelled monoclonal antibody anti-CD19-PE-Cy7, anti-CD3-APC and anti-CD45-APC-H7, or several tubes or recipients comprising separately or in combination each of these antibodies;
b. A tube or recipient comprising labelled monoclonal antibody anti-CD19-PE-Cy7, anti-CD5-APC and anti-CD45-APC-H7, or several tubes or recipients comprising separately or in combination each of these antibodies; and
c. Optionally a lysing Solution and/or a washing or buffer solution.

19. The kit of claim 12 or 13, wherein the means for determining the percentage of CD5+CD19+CD45+ B cells comprises the following tubes or recipients:
a. A tube or recipient comprising labelled monoclonal antibody anti-CD19-PE-Cy7, anti-CD3-APC and anti-CD45-APC-H7, or several tubes or recipients comprising separately or in combination each of these antibodies, wherein each of these monoclonal antibodies is found at a concentration between 0.02 and 1.5 μg/μl;
b. A tube or recipient comprising labelled monoclonal antibody anti-CD19-PE-Cy7, anti-CD5-APC and anti-CD45-APC-H7, or several tubes or recipients comprising separately or in combination each of these antibodies, wherein each of these monoclonal antibodies is found at a concentration between 0.02 and 1.5 μg/μl; and
c. Optionally a lysing Solution and/or a washing or buffer solution.

20. The kit of any of claims 13 to 19, wherein the kit comprises means for determining the percentage of CD45+CD8+perforin+CD56-CD3+ T cells and wherein said means comprise the following monoclonal antibodies: anti-CD8, anti-CD3, anti-CD56, anti-CD45 and FITC Anti-human Perforin Antibody.

21. The kit of claim 20, wherein the means for determining the percentage of CD45+CD8+perforin+CD56-CD3+ T cells comprises the following tubes or recipients:
a. A tube or recipient comprising anti-CD8, anti-CD3, anti-CD56 and anti-CD45, or several tubes or recipients comprising separately or in combination each of these antibodies;
b. A tube or recipient comprising FITC Anti-human Perforin Antibody; and
c. Optionally means for the fixation and permeabilization of cells and/or a buffer to wash the cells and to dilute the anti-cytokine antibodies for staining.

22. The kit of claim 20, wherein the means for determining the percentage of CD45+CD8+perforin+CD56-CD3+ T cells comprises the following tubes or recipients:
a. A tube or recipient comprising labelled monoclonal antibodies anti-CD8-PE, anti-CD3-PercP, anti-CD56-APC and anti-CD45-APC-H7, or several tubes or recipients comprising separately or in combination each of these antibodies;
b. A tube or recipient comprising FITC Anti-human Perforin Antibody; and
c. Optionally means for the fixation and permeabilization of cells and/or a buffer to wash the cells and to dilute the anti-cytokine antibodies for staining.

23. The kit of claim 20, wherein the means for determining the percentage of CD45+CD8+perforin+CD56-CD3+ T cells comprises the following tubes or recipients:
a. A tube or recipient comprising labelled monoclonal antibodies anti-CD8-PE, anti-CD3-PercP, anti-CD56-APC, anti-CD45-APC-H7 and an isotype control as for example IgG_{2b}-FITC (Anti dansyl), or several tubes or recipients comprising separately or in combination each of these antibodies;
b. A tube or recipient comprising FITC Anti-human Perforin Antibody;
c. A tube or recipient comprising anti-CD8-PE, anti-CD3-PercP, anti-CD56-APC and anti-CD45-APC-H7, or several tubes or recipients comprising separately or in combination each of these antibodies; and
d. Optionally means for the fixation and permeabilization of cells and/or a buffer to wash the cells and to dilute the anti-cytokine antibodies for staining.

24. The kit of claim 20, wherein the means for determining the percentage of CD45+CD8+perforin+CD56-CD3+ T cells comprises the following tubes or recipients:
a. A tube or recipient comprising monoclonal antibodies anti-CD8-PE, anti-CD3-PercP, anti-CD56-APC and anti-CD45-APC-H7, or several tubes or recipients comprising separately or in combination each of these antibodies, wherein each of these monoclonal antibodies is found at a concentration between 0.02 and 1.5 μg/μl;
b. A tube or recipient comprising FITC Anti-human Perforin Antibody; and
c. Optionally means for the fixation and permeabilization of cells and/or a buffer to wash the cells and to dilute the anti-cytokine antibodies for staining.

25. The kit of claim 20, wherein the means for determining the percentage of CD45+CD8+perforin+CD56-CD3+ T cells comprises the following tubes or recipients:
a. A tube or recipient comprising monoclonal antibodies anti-CD8-PE, anti-CD3-PercP, anti-CD56-APC, anti-CD45-APC-H7 and an isotype control as for example IgG_{2b}-FITC (Anti dansyl), or several tubes or recipients comprising separately or in combination each of these antibodies, wherein each of these monoclonal antibodies is found at a concentration between 0.02 and 1.5 μg/μl;
b. A tube or recipient comprising FITC Anti-human Perforin Antibody; and
c. A tube or recipient comprising labelled monoclonal antibody anti-CD8-PE, anti-CD3-PercP, anti-CD56-APC and anti-CD45-APC-H7, or several tubes or recipients comprising separately or in combination each of these antibodies, wherein each of these monoclonal antibodies is found at a concentration 0.02 and 1.5 μg/μl; and
d. Optionally means for the fixation and permeabilization of cells and/or a buffer to wash the cells and to dilute the anti-cytokine antibodies for staining.

26. The kit of any of claims 11 to 25, wherein the kit further comprises instructions for using said kit.

27. Use of the kit according to any of claims 12 to 19 for predicting response of a human subject to Interferon beta.
